(19) United States
(12) Patent Application Publication
(10) Pub. No.: US 2007/0202203 A1
(43) Pub. Date: Aug. 30, 2007

(54) ANTI-AGEING COMPOSITION

(75) Inventor: David AMAR, Paris (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 11/680,032

(22) Filed: Feb. 28, 2007

Related U.S. Application Data
(60) Provisional application No. 60/785,990, filed on Mar. 27, 2006.

(30) Foreign Application Priority Data
Feb. 28, 2006 (FR) 06 50706

(51) Int. Cl. A61K 38/04; A61K 36/48; A61K 31/728

(52) U.S. Cl. 424/757; 514/54; 514/19

(57) ABSTRACT

The invention relates to a composition containing, in a physiologically acceptable medium, at least: hyaluronic acid and/or one of its salts; at least one extract of soybean proteins; and at least one compound of formula (I) as described herein. This composition is useful for preventing and/or decreasing the signs of skin ageing, in particular the loss of firmness and/or of elasticity of the skin.

### REFERENCE TO PRIOR APPLICATIONS

This application claims priority to U.S. provisional application 60/785,990 filed Mar. 27, 2006, and to French patent application 0650706 filed Feb. 28, 2006, both incorporated herein by reference.

### FIELD OF THE INVENTION

The present invention relates to a composition comprising, in particular, at least:
hyaluronic acid and/or one of its salts;
an extract of soybean proteins; and
a compound of formula (I) as defined hereinafter, referred to in particular as pseudodipeptide in the rest of the description.

In particular, the pseudodipeptide is preferably {2-[acetyl-(3-trifluoromethylphenyl)amino]-3-methylbutyrylamino}acetic acid, otherwise called N-[N-acetyl, N′-(3-trifluoromethyl)phenylvalyl]glycine or N-acetyl-N-[3-(trifluoromethyl)phenyl]valylglycine or acetyl trifluoromethyl phenyl valylglycine, or an ester thereof with a C₁-C₆ alcohol.

The invention also relates to a combination intended to maintain and/or stimulate the proliferative activity of skin cells that have decreased with age, and also to a cosmetic process intended to prevent and/or decrease the signs of skin ageing using said composition.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

Women, and even men, currently have a tendency to wish to look youthful for as long as possible and consequently seek to fade out the age marks on the skin, which are reflected in particular by a loss of firmness and/or of elasticity and/or of tonicity and/or of suppleness of the skin and by the formation of wrinkles and fine lines. In this respect, the media and the fashion world report about products intended to keep the skin radiant and wrinkle-free for as long as possible, these being signs of youthful skin, and all the more so since the physical appearance acts on the psychi and/or on the morale.

Human skin consists of two compartments, namely a superficial compartment, the epidermis, and a deep compartment, the dermis.

Natural human epidermis is composed mainly of three types of cells, which are keratinocytes, that are very predominant, melanocytes and Langerhans cells. Each of these cell types contributes by virtue of its own functions to the essential role played in the organism by the skin.

The dermis provides the epidermis with a solid support. It is also its feeder element. It consists mainly of fibroblasts and of an extracellular matrix. Leucocytes, mast cells or tissue macrophages are also found therein. It also consists of blood vessels and of nerve fibres.

The extracellular matrix of the dermis is composed of proteins belonging to several major families. Collagens, matrix glycoproteins other than collagens (fibronectin, laminin), elastin, proteoglycans and glycosaminoglycans (GAGs) in free form (i.e. not bound to a protein), including hyaluronic acid. These proteins are predominantly synthesized by fibroblasts and it is known that the skin ageing process induces a decrease in these metabolic activities, resulting in a decrease in the dermal extracellular matrix proteins and a decrease in cell growth resulting in an impairment of the mechanical properties of the skin, in particular its firmness, its elasticity, its tonicity and/or its suppleness. Skin ageing is a natural physiological process; it is not considered to be a pathological condition or a therapeutic disorder.

There remains the need to have cosmetic compositions for preventing and/or decreasing the effects of ageing on the skin and/or restoring the mechanical properties of a younger skin, such as its firmness and/or its elasticity and/or its suppleness.

### SUMMARY OF THE INVENTION

Now, the inventor has discovered a combination that makes it possible to satisfy this need by preventing the decrease in and/or restoring the activity of protein synthesis in the cells of the dermis and/or restoring the proliferative activity of the skin cells. This combination comprises:
hyaluronic acid and/or one of its salts;
at least one extract of soybean proteins; and
at least one compound of formula (I) as defined hereinafter, referred to as a pseudodipeptide in the description.

In particular, this pseudodipeptide is preferably one or more of {2-[acetyl-(3-trifluoromethylphenyl)amino]-3-methylbutyrylamino}acetic acid, otherwise called N-[N-acetyl, N′-(3-trifluoromethylphenylvalyl]glycine or N-acetyl-N-[3-(trifluoromethyl)phenyl]valylglycine or acetyl trifluoromethyl phenyl valylglycine, or an ester of any thereof with a C₁-C₆ alcohol.

Hyaluronic acid is known for its effect on the moisturization of the skin. Cosmetic compositions comprising hydrolysed extracts of soybean are also known from the prior art. However, to the inventor's knowledge, specifically combining hyaluronic acid and/or one of its salts, at least one hydrolysed extract of soybean and at least one compound of formula (I) as defined hereinafter, in particular at least one of {2-[acetyl-(3-trifluoromethylphenyl)amino]-3-methylbutyrylamino}acetic acid, otherwise called N-[N-acetyl, N′-(3-trifluoromethyl)phenylvalyl]glycine or N-acetyl-N-[3-(trifluoromethyl)phenyl]valylglyine or acetyl trifluoromethyl phenyl valylglycine, or an ester of any thereof with a C₁-C₆ alcohol, had never been described or suggested up until now.

The present invention preferably relates to a composition comprising, in a physiologically acceptable medium, at least:
hyaluronic acid and/or one of its salts;
at least one extract of soybean proteins; and
at least one compound of formula (I): 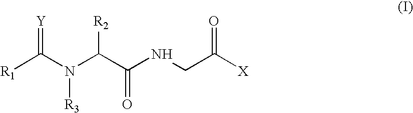
   in which:

the radical Y represents o,

the radical R1 represents a saturated or unsaturated, linear hydrocarbon-based radical having from 1 to 6 carbon atoms, preferably from 1 to 4 carbon atoms, and even more preferably from 1 to 2 carbon atoms, or a branched hydrocarbon-based radical having from 3 to 6 carbon atoms, preferably from 3 to 4 carbon atoms;

the radical R2 represents a saturated or unsaturated, linear hydrocarbon-based radical having from 1 to 10 carbon atoms, preferably having from 2 to 6 carbon atoms, or a branched hydrocarbon-based radical having from 3 to 10 carbon atoms, preferably having from 3 to 6 carbon atoms;

the radical R3 represents a phenyl radical optionally substituted with a saturated or unsaturated, linear or branched hydrocarbon-based radical having from 1 to 3 carbon atoms, optionally substituted with 1 or more fluorine atoms;

the radical X represents a radical chosen from —OH and —OR₄, preferably —OH, with R₄ representing a saturated or unsaturated, linear hydrocarbon-based radical having from 1 to 6 carbon atoms, or a branched hydrocarbon-based radical having from 3 to 6 carbon atoms.

In particular, the compound of formula (I) is preferably chosen from {2-[acetyl-(3-trifluoromethylphenyl)amino]-3-methylbutyryl-amino}acetic acid, otherwise called N-[N-acetyl, N′-(3-trifluoromethyl)phenylvalyl]glycine or N-acetyl-N-[3-(trifluoromethyl)phenyl]valylglycine or acetyl trifluoromethyl phenyl valylglycine, or an ester of any thereof with a C₁-C₆ alcohol.

Hyaluronic Acid

Hyaluronic acid belongs to the glycosaminoglycan (GAG) family. GAGs are linear chains composed of a repetition of a base diholoside always containing a hexosamine (glucosamine or galactosamine) and another monosaccharide (glucuronic acid, iduronic acid or galactose). Glucosamine is either N-sulphated or N-acetylated. On the other hand, galactosamine is always N-acetylated. In addition, there may be sulphates O-bonded to the hexosamine, uronic acid and galactose.

Hyaluronic acid or hyaluronan (HA) is the principal GAG of the dermis, the latter containing half the organism's HA. It is a polysaccharide of disaccharides that are themselves composed of D-glucuronic acid and of N-acetylglucosamine, linked to one another via alternating beta-1,4 and beta-1,3 glycosidic bonds. It plays an important role in the moisturization and the elasticity of the skin.

The composition according to the invention preferably contains hyaluronic acid and/or one of its salts. As hyaluronic acid salts, mention may in particular be made of potassium hyaluronate and sodium hyaluronate. Sodium hyaluronate will preferably be used according to the present invention.

The sodium hyaluronate with a molecular weight of 1,100,000 daltons, sold by the company SOLIANCE under the name CRISTALHYAL®, may in particular be used in the composition of the invention.

The hyaluronic acid or one of its salts is present in the composition according to the invention in any amount, preferably an amount ranging from 0.01% to 1% by weight (of solids) relative to the total weight of the composition, in particular from 0.01% to 0.1% by weight (of solids) relative to the total weight of the composition.

Extract of Soybean Proteins

The extract of soybean proteins used according to the invention may be any extract of soybean proteins that has a beneficial effect on the appearance and/or the mechanical properties of the skin, in particular on its firmness and/or its tonicity and/or its elasticity.

As extracts of soybean proteins that can be used in the composition according to the invention, mention may in particular be made of:
extracts of soybean that stimulate fibroblast proliferation, such as those sold by the company LSN under the name Eleseryl SH-VEG 8® or sold by the company SILAB under the trade name Raffermine®;
extracts of soybean that increase the synthesis of collagen and/or prevent the degradation thereof, such as the soybean hydrolysate sold by the company COLETICA under the trade name Phytokine®, or the hydrolysed extract of soybean proteins sold by the company SILAB under the trade name RIDULISSE C®.

A hydrolysed extract of soybean proteins that increases the synthesis of collagen and/or prevents the degradation thereof will preferably be used. The ability of an extract of soybean proteins to increase the synthesis of collagen and/or prevent the degradation thereof can be evaluated in vitro, for example, according to the following method, without undue effort in view of this teaching:
human fibroblasts in culture are incubated in the presence or absence (control) of an extract of soybean proteins to be tested or in the presence of a reference product known to increase the synthesis of collagen, such as vitamin C (positive control);
the cell supernatants are recovered;
the collagens are assayed by an ELISA method;
the extracts for which an increase in the synthesis of collagen, compared to the control, is obtained are selected.

In particular, the extract of soybean proteins used in the composition according to the invention preferably contains a product A obtained from soybean cake and a product B obtained from soybean fibres, said products being respectively obtained by means of an extraction process comprising at least one solubilization step, a protease hydrolysis, a step for separating the solubilized phase and a step for concentrating the active phase.

A hydrolysed extract of soybean proteins that is preferred according to the invention is the extract prepared according to the method described in Application WO2004/096168 from SILAB and sold by said company under the name RIDULISSE C®.

The method described in Application WO2004/096168 consists in combining two products A and B, obtained from soybean, each being obtained by means of the succession of the following steps:

Product A
solubilization of soybean cake in a proportion of at least 50 g/l by weight, in water at an acidic pH,
simultaneous or successive enzymatic hydrolyses in the presence of proteases,
filtration, decanting or centrifugation in order to separate the solubilized phase and the insoluble products, and
successive concentrating of the active phase by filtration, ultrafiltration, osmosis or nanofiltration;

Product B
solubilization of soybean fibres in water at an acidic pH,
at least one enzymatic hydrolysis using at least one protease,
filtration, decanting or centrifugation in order to separate the solubilized phase and the insoluble products, and
successive concentrating of the active phase by filtration, ultrafiltration, osmosis or nanofiltration.

The two products A and B are then combined; at least 10% of the product B is preferably combined with the product A.

The amount of extract of soybean proteins used in the composition according to the invention is not limited and is preferably an amount required to obtain the desired beneficial effect on the skin, in particular an effect on the firmness and/or the elasticity of the skin. By way of example, the composition contains from 0.01% to 5% of extract of soybean proteins by weight relative to the total weight of the composition, preferably from 0.05% to 2% by weight relative to the total weight of the composition, and even more preferably from 0.1% to 1% by weight relative to the total weight of the composition.

Pseudodipeptide

The compound used in the composition according to the invention, also called pseudodipeptide in the rest of the description corresponds to formula (I) below: 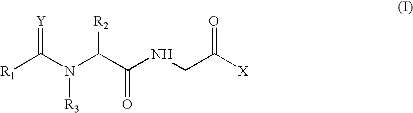
in which:
the radical Y represents O,
the radical R1 represents a saturated or unsaturated, linear hydrocarbon-based radical having from 1 to 6 carbon atoms, preferably from 1 to 4 carbon atoms, even more preferably from 1 to 2 carbon atoms, or a branched hydrocarbon-based radical having from 3 to 6 carbon atoms, preferably from 3 to 4 carbon atoms;
the radical R2 represents a saturated or unsaturated, linear hydrocarbon-based radical having from 1 to 10 carbon atoms, preferably having from 2 to 6 carbon atoms, or a branched hydrocarbon-based radical having from 3 to 10 carbon atoms, preferably having from 3 to 6 carbon atoms;
the radical R3 represents a phenyl radical optionally substituted with a saturated or unsaturated, linear or branched hydrocarbon-based radical having from 1 to 3 carbon atoms, optionally substituted with 1 or more fluorine atoms;
the radical X represents a radical chosen from —OH and —OR₄, preferably —OH, with R₄ representing a saturated or unsaturated, linear hydrocarbon-based radical having from 1 to 6 carbon atoms, or a branched hydrocarbon-based radical having from 3 to 6 carbon atoms.

One preferred saturated or unsaturated, linear or branched hydrocarbon-based radical is an alkyl group.

These compounds are described in the Applicant's patent EP 1 292 608 B1, incorporated herein by reference.

In particular, the compound is a glycine derivative.

Among the preferred compounds, specific mention may be made of: {2-[acetyl-(3-trifluoromethylphenyl)amino]-3-methylbutyrylamino}acetic acid, otherwise called N-[N-acetyl, N′-(3-trifluoromethyl)phenylvalyl]glycine or N-acetyl-N-[3-(trifluoromethyl)phenyl]valylglycine or acetyl trifluoromethyl phenyl valylglycine; ethyl {2-[acetyl-(3-trifluoromethylphenyl)amino]-3-methylbutyrylamino}acetate, otherwise called N-acetyl-N-[3-(trifluoromethyl)phenyl]valylglycine ethyl ester; [2-(acetylbenzylamino)-3-methylbutyrylamino]acetic acid; ethyl [2-(acetylbenzylamino)-3-methylbutyrylamino]-acetate; ethyl (2-{benzyl[(diethoxyphosphoryl)acetyl]amino}-3-methylbutyrylamino)acetate.

A compound that is particularly preferred according to the invention is chosen from {2-[acetyl-(3-trifluoromethylphenyl)amino]-3-methylbutyrylamino}-acetic acid, otherwise called N-[N-acetyl, N′-(3-trifluoromethyl)phenylvalyl]glycine or N-acetyl-N-[3-(trifluoromethyl)phenyl]valylglycine or acetyl trifluoromethyl phenyl valylglycine, and esters thereof with C₁-C₆ lower alcohols.

As esters, mention may in particular be made of its ethyl ester, ethyl {2-[acetyl-(3-trifluoromethylphenyl)amino]-3-methylbutyrylamino}-acetate, otherwise called N-acetyl-N-[3-(trifluoromethyl)phenyl]valylglycine ethyl ester.

These compounds are described, in patent EP 1 292 608 B1, as elastase inhibitors and/or agents for preventing elastin degradation.

The composition according to the invention may contain any amount, preferably from 0.01% to 10% by weight of the one or more compounds of formula (I) relative to the total weight of the composition, and preferably from 0.1% to 2% by weight relative to the total weight of the composition.

According to a preferred embodiment of the invention, the composition according to the invention may also comprise adenosine or a manganese salt, in particular manganese gluconate.

The inventor has effectively shown, on a model of contractile latices, that this combination has a dermo-decontracting effect that can be taken advantage of in the anti-ageing compositions according to the invention. This combination in fact makes it possible to prevent and/or decrease dermal tensing of the facial skin that is responsible in particular for expression wrinkles.

These expression wrinkles are produced under the effect of the stress exerted on the skin by the skin muscles that allow facial expressions. Depending on the shape of the face, the frequency of facial expressions and possible ticks, they may appear even from childhood. Age, and also certain environmental factors such as exposure to sunlight, do not play a part in generating them, but may make them deeper and permanent.

Expression wrinkles are characterized by the presence of grooves around the orifices formed by the nose (nasal grooves), the mouth (perioral wrinkles and “sour-face” wrinkles) and the eyes (crows'-feet wrinkles), around which are the skin muscles, and also between the eyebrows (glabella wrinkles or lion wrinkles) and on the forehead.

The composition according to the invention preferably contains from 0.01% to 1% of adenosine by weight relative to the total weight of the composition, more preferably from 0.01% to 0.1% by weight relative to the total weight of the composition.

The term “manganese salts” is intended to mean organic or inorganic manganese salts.

As organic manganese salts, mention may be made of manganese gluconate, manganese carbonate, manganese acetate, manganese citrate, manganese oleate or manganese oxalate.

As inorganic manganese salts, mention may be made of the mineral salts such as manganese chloride, manganese borate, manganese nitrate, manganese phosphate or manganese sulphate.

Manganese gluconate will preferably be used.

The composition according to the invention thus preferably contains from 0.01 to 1% of manganese salt by weight relative to the total weight of the composition, more preferably from 0.01% to 0.1% by weight, relative to the total weight of the composition.

The composition according to the invention may also comprise at least one active agent chosen from vitamins and derivatives thereof. In particular, the vitamins and derivatives are chosen from tocopherol or tocopheryl acetate.

Galenics

The composition according to the invention may be in any galenic form inclusding those conventionally used for topical application, and in particular in the form of aqueous gels of the serum type, or of aqueous or aqueous-alcoholic solutions. It may also, through the addition of a fatty or oily phase, be in the form of a dispersion of the lotion or serum type, an emulsion with a liquid or semi-liquid consistency of the milk type, obtained by dispersion of a fatty phase in an aqueous phase (O/W emulsion) or conversely (W/O emulsion), or a suspension or emulsion with a soft, semi-solid or solid consistency of the cream or gel type, or else a multiple emulsion (W/O/W or O/W/O emulsion), a microemulsion, a vesicular dispersion of ionic and/or non-ionic type or a wax/aqueous phase dispersion. These compositions are prepared according to the usual methods.

According to a preferred embodiment of the invention, the composition is in the form of an oil-in-water emulsion.

When the composition is in the form of an emulsion, the proportion of the oily phase of the emulsion may range, for example, from 1% to 80% by weight, and preferably from 5% to 40% by weight, relative to the total weight of the composition. The oils, the emulsifiers and the co-emulsifiers used in the composition in the form of an emulsion are chosen from those conventionally used in the cosmetics or dermatological field. The emulsifier and the co-emulsifier are generally present in the composition in a proportion ranging from 0.3% to 30% by weight, and preferably from 0.5% to 20% by weight, relative to the total weight of the composition. The emulsion may also contain lipid vesicles.

As fatty substances that can be used in the invention, use may be made of oils, and in particular hydrocarbons of mineral or synthetic origin (liquid petroleum jelly, hydrogenated polyisobutene, mineral oil), oils of plant origin (shea butter, avocado oil, soybean oil, apricot kernel oil), oils of animal origin (lanolin), synthetic esters (pentaerythrityl tetraoctanoate, 2-ethylhexyl palmitate, myristyl myristate), silicone oils (polydimethylsiloxane and cyclopentasiloxane) and fluoro oils (perfluoropolyethers). Fatty alcohols such as cetyl alcohol or stearyl alcohol, fatty acids such as stearic acid, waxes such as beeswax and gums, in particular silicone gums, can also be used as fatty substances.

As emulsifiers and co-emulsifiers that can be used in the invention, mention may, for example, be made of fatty acid esters of polyethylene glycol, such as PEG-100 stearate and PEG-20 stearate; optionally oxyethylenated fatty acid esters of alkylglycoside, such as oxyethylenated (20 OE) methylglucose sesquistearate; esters of a fatty acid and of a polyol, such as glyceryl stearate, sorbitan tristearate and the oxyethylenated sorbitan stearates available under the trade names Tween® 20 or Tween® 60, for example; and mixtures thereof.

As hydrophilic gelling agents, mention may in particular be made of carboxyvinyl polymers (carbomer), acrylic copolymers such as acrylate/alkyl acrylate copolymers, polyacrylamides optionally in the form of copolymers in an inverse emulsion, such as the products sold, respectively, by CLARIANT under the trade name HOSTACERIN AMPS and by SEPPIC under the trade name SEPIGEL 305, polysaccharides, such as xanthan gum, and clays, and as lipophilic gelling agents, mention may be made of modified clays such as bentones, metal salts of fatty acids, and hydrophobic silica.

As preserving agents, mention may be made of para-hydroxybenzoic acid esters, octane-1,2-diol, 3-iodo-2-propynylbutyl carbamate, phenoxyethanol, imidazolinyl-urea and chlorhexidine digluconate.

As fillers, mention may, for example, be made of polyamide (Nylon®) particles; poly(methyl methacrylate) microspheres; ethylene-acrylate copolymer powders; expanded powders such as hollow microspheres, and in particular the microspheres formed from a terpolymer of vinylidene chloride, acrylonitrile and methacrylate and sold under the name EXPANCEL by the company Kemanord Plast; powders of natural organic materials, such as starch powders, in particular powders of maize starch, of wheat starch or of rice starch, which may or may not be crosslinked, for instance powders of starch crosslinked with octenylsuccinate anhydride; silicone resin microbeads such as those sold under the name TOSPEARL by the company Toshiba Silicone; talc; silica; metal oxides such as titanium dioxide or zinc oxide; mica; and mixtures thereof.

The invention also relates to the use of the composition according to the invention to prevent and/or decrease thinning of the skin and/or to promote cell renewal.

Other uses of the composition according to the invention include to stimulate the proliferative activity of skin cells that has decreased with age, to maintain and/or stimulate skin cell proliferation, to promote the resumption of skin cell proliferation that has in particular been impaired in the course of ageing, to maintain and/or stimulate synthesis of the collagen and/or elastin fibres of the skin tissue, to reassemble and/or repair the fibres of the skin tissue that have become impaired with age, and to repair wrinkles from the inside. Generally, the amount of the invention composition is that amount that effects such result(s), or other results discussed herein, and generally the composition of the invention is applied to skin in need of such benefit/repair.

The compound of formula (I) preferably includes at least one of {2-[acetyl-(3-trifluoromethylphenyl)amino]-3-methylbutyryl-amino}acetic acid, otherwise called N-[N-acetyl, N′-(3-trifluoromethyl)phenylvalyl]glycine or N-acetyl-N-[3-(trifluoromethyl)phenyl]valylglycine or acetyl trifluoromethyl phenyl valylglycine, and esters of any thereof with C₁-C₆ lower alcohols.

The invention compositions make it possible, for example via an effect on the synthesis of elastin and/or the prevention of the degradation thereof and/or on the synthesis of collagen and/or the prevention of the degradation thereof, and also an effect on cell proliferation, to decrease wrinkles by promoting and/or enhancing the fibres of the skin tissue that have become impaired in the course of ageing.

According to a preferred embodiment of the invention, the use of the three preferred active agents described above is combined in the same composition with adenosine and manganese gluconate, the second combination and/or the composition being intended to prevent and/or decrease dermal tensing of the facial skin.

This supplementary combination also makes it possible to prevent and/or decrease expression wrinkles on the face.

The invention also relates to a process for preventing and/or correcting the cutaneous signs of ageing, in particular wrinkles and fine lines and/or the loss of firmness and/or the elasticity of the skin, comprising the topical application to the skin of a composition according to the invention comprising at least the complex of 3 active agents defined above.

In particular, when the composition according to the invention also comprises adenosine and a manganese salt, the process according to the invention also makes it possible to prevent and/or decrease dermal tensing of the skin, and thus to prevent and/or reduce expression wrinkles.

The wrinkles treated according to the process of the invention are in particular the wrinkles lying radially around the mouth and/or the eyes and/or horizontally on the forehead and/or located in the gap between the eyebrows.

Preferably, the composition according to the invention is applied to wrinkled skin.

In particular, it is applied to areas where there is sagging of the skin and/or the formation of wrinkles, in particular to areas of the forehead that are marked with expression wrinkles and/or on individuals with expression wrinkles.

The composition may be applied daily to the skin of the face and/or of the neck, in particular to the areas identified above.

The invention will now be illustrated by means of the following non-limiting examples.

### EXAMPLES

### Example 1

### Anti-Ageing Effect of the Complex of 3 Active Agents on Skin Maintained Under Survival Conditions and Aged Experimentally

a) Protocol

The effect of the mixture of three active agents according to the invention on skin ageing is studied. The test is carried out on a model of human skin maintained under survival conditions and aged experimentally. This model of experimental ageing is realised by topical application of a class II dermocorticoid (Diprosone®, cream containing betamethasone at 0.05%) at D0, D1 and D2 in order to obtain a decrease in the mitotic index (cell proliferation of the skin cells) and an impairment of the metabolism of the fibroblasts responsible for synthesis of the macromolecules of the connective tissue, in particular collagen and elastin.

The mixture of the following 3 active agents is tested:

| Composition of the test mixture | Final concentration tested |
|---|---|
| Acetyl trifluoromethylphenyl | 0.10% |
| valylglycine* | |
| Hydrolysed extract of soybean | 2.00% |
| (RIDULISSE C ® sold by SILAB) | |
| Sodium hyaluronate | 0.02% |

*= {2-[acetyl-(3-trifluoromethylphenyl)amino]-3-methylbutyrylamino}acetic acid, otherwise called N-[N-acetyl, N′-(3-trifluoromethyl)phenylvalyl]glycine or N-acetyl-N-[3-(trifluoromethyl)phenyl]valylglycine or acetyl trifluoromethyl phenyl valylglycine

The treatment with the combination will be carried out secondarily for 13 days, from D1.

The analysis of cell proliferation is carried out at D3 and the collagen, elastin and glycosaminoglycan (GAG) assays at D15.

b) Results

The epithelial proliferation is analysed at D3 by immunohistochemistry using an anti-Ki67 antibody (marker for cells in the M, S, G1 and G2 phases of the cell cycle). The collagen, elastin and GAG assays are carried out by means of a spectrocolorimetric assay method (Sircol Collagen Assay, Interchim). The results are related to the total amount of proteins, measured at 562 nm (BCA assay, Pierce); the results are expressed in ng of collagen or elastin or GAGs/mg of proteins.

The statistical analysis is carried out by means of the paired Student's test.

The results are presented in the following table:

| | % epithelial | | | |
|---|---|---|---|---|
| | cells labelled | | | |
| | with the Ki67 | Collagen | Elastin | GAGs |
| Treatment | antibody | (μg/mg of proteins) | (μg/mg of proteins) | (μg/mg of proteins) |
| Control skin | 6.1 | 117.9 | 136.8 | 16.9 |
| Skin + | 1.78 | 92.4 | 84.5 | 14.6 |
| dermocorticoid | | | | |
| Skin + | 6.8 | 122 | 113.4 | 18.7 |
| dermocorticoid + | | | | |
| mixture of the | | | | |
| 3 active agents | | | | |

These results show that the treatment of human skin under survival conditions, with a dermocorticoid, induces a decrease in cell proliferation, and also in collagen and elastin synthesis. This treatment has no effect on the production of GAGs. This type of treatment makes it possible to age the skin experimentally.

The treatment of experimentally aged human skin with the mixture of the active agents induces a resumption of cell proliferation and also a significant increase in collagen and elastin synthesis.

This effect of the combination of the 3 active agents according to the invention on this model of aged skin can be taken advantage of in anti-ageing compositions intended to maintain and/or stimulate the proliferative and protein synthesis activity of skin cells that has become impaired in the course of ageing.

### Example 2

### Dermodecontracting Effect of the Combination of Adenosine and Manganese Gluconate

a) Principle of the Test

The principle of this test consisted in studying the dermodecontracting effect of a combination of adenosine and manganese gluconate on a model of dermal equivalent consisting of a collagen matrix seeded with normal human fibroblasts.

These conditions are intended to mimic in vitro, the dermal contractile phenomena that occur during dermal tensing of the face, for example during facial expressions. Under these conditions, in fact, the cells spontaneously express tensile forces that induce retraction of the collagen gel. This results in a decrease of the total surface area of the dermal equivalent over time. The measurement of this surface area makes it possible to evalue the relaxing or dermocontracting effects of the substances previously brought into contact with the dermal equivalent.

b) Protocol

Series of three attached dermal equivalents containing normal human fibroblasts are prepared: a control series with no treatment; three series respectively treated with adenosine (0.004%), manganese gluconate (0.0005%) and the mixture of these two compounds in the proportions indicated. The experiment is repeated three times.

The dermal equivalents are prepared as described in Asselineau et al., Exp. Cell. Res., 1985, 159, 536-539; Models in dermatology, 1987, vol 3 pp 1-7, in the following proportions:

| | |
|---|---|
| MEM medium (1.76X) with or without test compound(s) | 45% |
| Foetal calf serum: | 9% |
| NaOH (0.1 N): | 5% |
| Acetic acid (1/1000): | 4% |
| Collagen: | 26% |
| Fibroblasts: | 11% |

The collagen used is collagen type I (commercial solution), but collagen type III or IV can also be used. It is extracted from rat tails or from calf skin by acid hydrolysis and conserved in an acidic medium at +40° C.; it polymerizes naturally by heating to 37° C. and by decreasing the degree of acidity. The collagen is dialysed beforehand against successive baths of water +acetic acid.

The protocol is the following: the 1.76×MEM medium, in the presence of additives (1% glutamine, 1% nonesssential amino acids, 1% sodium pyruvate, 1% fungizone and 1% pencillin/streptomycin), the foetal calf serum and the 0.1 N sodium hydroxide NaOH are introduced into a sterile falcon tube. The fibroblasts, isolated from human skin explants, are then added at the concentration of 1.4×10⁵ cells per 1 ml of culture medium.

A volume/volume mixture of collagen in acetic acid at 1/1000 is then added slowly, against the wall of the tube so as to observe the appearance of a whitish cloud.

The whole is then mixed carefully and dispensed into the wells of a 12-well culture plate (Costar type, reference 3512) at a rate of 0.5 ml of mixture per cm². The culture plate is then placed in an incubator at 37° C. with 5% CO2.

Once formed after polymerization of the collagen, the dermal equivalents are left adhered to the culture support for 3 days and then detached from the support so that the contraction can begin. These attached dermal equivalents are taken out of the incubator in order to take images with a view to measuring their surface area after 24 h of contraction.

The evaluation of the spontaneous contraction of the treated and control dermal equivalents is carried out by measuring their surface area at 24 h after the beginning of the spontaneous contraction.

For this, a digital image is acquired for each treated or nontreated dermal equivalent by means of a camera (CCD camera—Iris Sony DXC—107P) and the surface area is then calculated on each image by means of an image analysis system (Zeiss Axiovision 3.0). This surface area measurement corresponds to a percentage contraction equal to the ratio of the surface areas according to the formula:
% contraction=(Sp−Si)/Sp×100
where:

Sp represents the surface area of a well of the culture plate; it corresponds to the total surface area of the dermal equivalent before contraction,

Si represents the surface area of the dermal equivalent at contraction time 24 h.

c) Results

| Treatment | % contraction |
|---|---|
| Control | 100 |
| Manganese gluconate (0.0005%) | 89 |
| Adenosine (0.004%) | 82 |
| Mn gluconate (0.0005%) + adenosine | 72 |
| (0.004%) | |

These results show that the combination of adenosine and manganese gluconate results in a delay of contraction of 28%, greater than that obtained with each of the compounds taken separately.

This test thus demonstrates that the combination of adenosine and manganese gluconate leads to less contraction of the dermal equivalent, compared to the control and to the dermal equivalent treated with these compounds separately.

This dermodecontracting effect of the combination of adenosine and manganese gluconate can be taken advantage of in the anti-ageing compositions of the invention, in particular for decreasing and/or limiting dermal tensing of the facial skin, and thus smoothing the wrinkles, in particular the expression wrinkles on the face.

### Example 3

### Formulation

This composition is prepared in a manner conventional for those skilled in the art. The amounts given in this example are indicated as percentages by weight.

Cream (oil-in-water emulsion)

| Acetyl trifluoromethylphenyl valylglycine* | | 0.30% |
|---|---|---|
| Hydrolysed soybean protein | | 0.10% |
| (Ridulisse C ® from SILAB | | |
| Sodium hyaluronate (Cristalhyal ® from SOLIANCE) | | 0.05% |
| Manganese gluconate | | 0.05% |
| Adenosine | | 0.04% |
| Talc | | 1.10% |
| Silica | | 1.20% |
| Octyldodecanol | | 0.50% |
| Isostearyl neopentanoate | | 3.50% |
| Apricot kernel oil | | 3.00% |
| Plant oils | | 3.30% |
| Squalene | | 0.90% |
| Cetyl alcohol | | 0.50% |
| Stearyl alcohol | | 0.50% |
| Hydrogenated polyisobutene | | 6.50% |
| (Perleam ® from NOF Corp) | | |
| Ammonium polyacryloyldimethyl taurate | | 1.00% |
| (Hostacerin AMPS ® from CLARIANT) | | |
| Polyamide fibres | | 3.00% |
| Cyclohexasiloxane | | 7.00% |
| Silicone film-forming polymer in the form of | | 2.00% |
| an emulsion (HMW2220 ® from DOW CORNING) | | |
| Tocopheryl acetate | | 0.50% |
| Glycerol | | 3.00% |
| Triethanolamine | | 0.15% |
| Emulsifiers | | 5.80% |
| Fragrances | | 0.50% |
| Preserving agents | | 1.30% |
| Water | qs | 100% |

*= {2-[acetyl-(3-trifluoromethylphenyl)amino]-3-methyl-butyrylamino}acetic acid, otherwise called N-[N-acetyl, N′-(3-trifluoromethyl)phenylvalyl]glycine or N-acetyl-N-[3-(trifluoromethyl)phenyl]valylglycine or acetyl trifluoromethyl phenyl valylglycine, prepared as described in patent EP 1 292 608 B1.

This cream can be applied daily to the face in the morning and/or the evening in order to reduce wrinkles and limit dermal tensing of the skin.

The above written description of the invention provides a manner and process of making and using it such that any person skilled in this art is enabled to make and use the same, this enablement being provided in particular for the subject matter of the appended claims, which make up a part of the original description and including a composition comprising, in a physiologically acceptable medium, at least:
hyaluronic acid and/or one of its salts;
at least one extract of soybean proteins; and
at least one compound of formula (I): 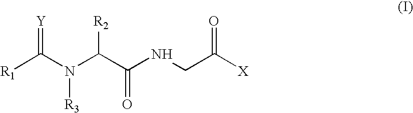
   in which:
the radical Y represents O,
the radical R1 represents a saturated or unsaturated, linear hydrocarbon-based radical having from 1 to 6 carbon atoms, preferably from 1 to 4 carbon atoms, and even more preferably from 1 to 2 carbon atoms, or a branched hydrocarbon-based radical having from 3 to 6 carbon atoms, preferably from 3 to 4 carbon atoms;
the radical R2 represents a saturated or unsaturated, linear hydrocarbon-based radical having from 1 to 10 carbon atoms, preferably having from 2 to 6 carbon atoms, or a branched hydrocarbon-based radical having from 3 to 10 carbon atoms, preferably having from 3 to 6 carbon atoms;
the radical R3 represents a phenyl radical optionally substituted with a saturated or unsaturated, linear or branched hydrocarbon-based radical having from 1 to 3 carbon atoms, optionally substituted with 1 or more fluorine atoms; and
the radical X represents a radical chosen from —OH and —OR₄, preferably —OH, with R₄ representing a saturated or unsaturated, linear hydrocarbon-based radical having from 1 to 6 carbon atoms, or a branched hydrocarbon-based radical having from 3 to 6 carbon atoms.

As used herein, the phrases “selected from the group consisting of,” “chosen from,” and the like include mixtures of the specified materials. Terms such as “contain(s)” and the like as used herein are open terms meaning ‘including at least’ unless otherwise specifically noted.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

The invention method and composition is preferably used by subjects desirous of the benefits noted herein, subjects “in need of” these benefits. Such subjects are typically suffering from wrinkles, or wish to stimulate the proliferative activity of skin cells that has decreased with age, to maintain and/or stimulate skin cell proliferation, to promote the resumption of skin cell proliferation that has in particular been impaired in the course of ageing, to maintain and/or stimulate synthesis of the collagen and/or elastin fibres of the skin tissue, to reassemble and/or repair the fibres of the skin tissue, etc., as explained above. In this regard, the invention process can be viewed as one for delaying the onset of the appearance of, and/or for reducing signs of, ageing of the skin.

Naturally, one using the invention as disclosed will use an amount of the invention composition effective to accomplish the intended result(s). Such amount is inclusive of an amount of the compositions described herein at the disclosed concentrations of active ingredients sufficient to cover the area of the skin being treated in a single application, and of course includes that amount applied upon repeated application, for example on a daily basis over a course of days, weeks, etc. In a preferred embodiment the invention process includes multiple applications of the invention composition to the area(s) of skin in need of attention.

1. A composition comprising, in a physiologically acceptable medium, at least:
hyaluronic acid and/or one of its salts;
at least one extract of soybean proteins; and
at least one compound of formula (I): 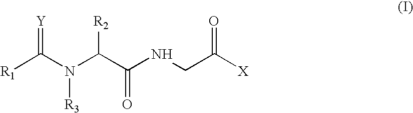
in which:
Y represents O,
R1 represents a saturated or unsaturated, linear hydrocarbon-based radical having from 1 to 6 carbon atoms or a branched hydrocarbon-based radical having from 3 to 6 carbon atoms;
R2 represents a saturated or unsaturated, linear hydrocarbon-based radical having from 1 to 10 carbon atoms or a branched hydrocarbon-based radical having from 3 to 10 carbon atoms;
R3 represents a phenyl radical optionally substituted with a saturated or unsaturated, linear or branched hydrocarbon-based radical having from 1 to 3 carbon atoms, optionally substituted with 1 or more fluorine atoms; and
the radical X represents a radical chosen from —OH and —OR₄, with R₄ representing a saturated or unsaturated, linear hydrocarbon-based radical having from 1 to 6 carbon atoms, or a branched hydrocarbon-based radical having from 3 to 6 carbon atoms.

2. The composition according to claim 1, comprising N-acetyl-N-[3-(trifluoromethyl)phenyl]valylglycine and/or an ester thereof with a C₁-C₆ alcohol.

3. The composition according to claim 1, comprising sodium hyaluronate.

4. The composition according to claim 1, wherein the extract of soybean proteins comprises a product A obtained from soybean cake and a product B obtained from soybean fibres, said products A and B being respectively obtained by an extraction process comprising at least one solubilization, protease hydrolysis, separating the solubilized phase, and concentrating the active phase.

5. The composition according to claim 1, comprising 0.01% to 1% by weight of hyaluronic acid and/or one of its salts, relative to the total weight of the composition.

6. The composition according to claim 1, comprising from 0.05% to 2% by weight of extract of soybean proteins, relative to the total weight of the composition.

7. The composition according to claim 1, comprising from 0.1% to 2% by weight of at least one compound of formula (I), relative to the total weight of the composition.

8. The composition according to claim 1, further comprising adenosine and a manganese salt.

9. The composition according to claim 8, comprising manganese gluconate.

10. A method to prevent and/or decrease thinning of the skin and/or to promote cell renewal, to stimulate the proliferative activity of skin cells that has decreased with age, to maintain and/or stimulate synthesis of the collagen and/or elastin fibres of the skin tissue, and/or to reassemble and/or repair the fibres of the skin tissue that have become impaired with age, comprising applying the composition of claim 1 to skin in need thereof.

11. A method to prevent and/or decrease dermal tensing of the facial skin comprising applying the composition of claim 1 to skin in need thereof, said composition further comprising adenosine and manganese gluconate.

12. A method for preventing and/or decreasing the cutaneous signs of ageing, preventing and/or decreasing wrinkles and fine lines and/or the loss of firmness and/or of elasticity of the skin, and/or for preventing and/or reducing expression wrinkles, comprising applying the composition of claim 1 to skin in need thereof.

13. The method of claim 12, wherein the wrinkles are the wrinkles lying radially around the mouth and/or the eyes and/or horizontally on the forehead and/or located in the gap between the eyebrows.

14. The method of claim 12, wherein the composition is applied to areas of the face or of the forehead that are marked with expression wrinkles.
